(19) United States
(12) Patent Application Publication
(10) Pub. No.: US 2019/0234935 A1
(43) Pub. Date: Aug. 1, 2019

(54) METHODS FOR SCREENING FOR MODULATORS OF GDF15-LIKE BIOLOGICAL ACTIVITY

(71) Applicant: Janssen Biotech, Inc., Horsham, PA (US)

(72) Inventors: Anthony Armstrong, Lawrence Township, NJ (US); Stephen Beck, Collegeville, PA (US); Jose Antonio Chavez, Doylestown, PA (US); Chen-Ni Chin, Devon, PA (US); Thai Dinh, Solana Beach, CA (US); Jennifer Furman, San Diego, CA (US); Matt Husovsky, Ramona, CA (US); Xiefan Lin-Schmidt, Ambler, PA (US); Shannon Mullican, Philadelphia, PA (US); Shamina Rangwala, London (GB); Vicki South, Collegeville, PA (US)

(21) Appl. No.: 16/341,074

(22) PCT Filed: Oct. 11, 2017
(86) PCT No.: PCT/US2017/056069

§ 371 Date: Apr. 11, 2019

Related U.S. Application Data
(60) Provisional application No. 62/407,046, filed on Oct. 12, 2016.

(51) Int. Cl. G01N 33/50; A61K 49/00

(52) U.S. Cl. CPC G01N 33/502; G01N 2333/495; A61K 49/0008

(57) ABSTRACT

A novel receptor for GDF15 was identified (GFRAL), as well as the use of this receptor in the identification or screening of GDF15 agonists or antagonists. These agonist or antagonist compounds may be used to either potentiate or suppress GDF15-like effects, respectively, at the cellular and organism levels, and may be used in treatment of metabolic diseases, including obesity, type 2 diabetes, hyperglycemia, hyperinsulinemia, dyslipidemia, diabetic nephropathy, or anorexia.

### FIELD OF THE INVENTION

The invention relates generally to the field of metabolic disorder drug research. More particularly, the invention relates to methods for identifying compounds that are capable of either agonizing or antagonizing GDF15, and kits for practicing these methods.

### BACKGROUND OF THE INVENTION

GDF15, a member of the TGFβ family, is a secreted protein that circulates in plasma as a 25 kDa homodimer. Plasma levels of GDF15 range between 150 and 1150 pg/ml in most individuals (Tsai et al., J Cachexia Sarcopenia Muscle. 2012, 3: 239-243). Plasma levels of GDF15 are increased under conditions of injury, cardiovascular disease and certain types of cancer. This upregulation is thought to be a cytoprotective mechanism. High plasma levels of GDF15 are associated with weight loss due to anorexia and cachexia in cancer, and in renal and heart failure. In a clinical trial, GDF15 levels were an independent predictor of insulin resistance in obese, non-diabetic subjects (Kempf et al., Eur. J. Endo. 2012, 167: 671-678). A study in twins showed that the differences in levels of GDF15 within twin pairs correlated to the differences in BMI within that pair, suggesting that GDF15 serves as a long-term regulator of energy homeostasis (Tsai et al., PLoS One. 2015, 10(7):e0133362).

While GDF15 has been extensively studied as a biomarker for several cardiovascular and other disease states, a protective role for GDF15 has also been described in myocardial hypertrophy and ischemic injury (Collinson, Curr. Opin. Cardiol. 2014, 29: 366-371; Kempf et al., Nat. Med. 2011, 17: 581-589; Xu et al., Circ Res. 2006, 98:342-50). GDF15 was shown to play an important role in protection from renal tubular and interstitial damage in mouse models of type 1 and type 2 diabetes (Mazagova et al., Am. J Physiol. Renal Physiol. 2013; 305: F1249-F1264). GDF15 is proposed to have a protective effect against age-related sensory and motor neuron loss, and it improves recovery consequent to peripheral nerve damage (Strelau et al., J. Neurosci. 2009, 29: 13640-13648; Mensching et al., Cell Tissue Res. 2012, 350: 225-238). In fact, GDF15 transgenic mice were shown to have a longer lifespan than their littermate controls, which can indicate that this molecule provides and advantage as a long-term survival factor (Wang et al., Aging. 2014, 6: 690-700).

Numerous reports have demonstrated the improvement of glucose tolerance and insulin sensitivity in mouse models upon treatment with GDF15 protein. Two independent strains of transgenic mice overexpressing GDF15 have decreased body weight and fat mass, as well as improved glucose tolerance (Johnen et al., Nat. Med. 2007, 13:1333-1340; Macia et al., PLoS One. 2012, 7:e34868; Chrysovergis et al., Int. J. Obesity. 2014, 38: 1555-1564). Increases in whole-body energy expenditure and oxidative metabolism were reported in GDF15 transgenic mice (Chrysovergis et al., 2014, Id.). These were accompanied by an increase in thermogenic gene expression in brown adipose tissue and an increase in lipolytic gene expression in white adipose tissue. Mice lacking the GDF15 gene have increased body weight and fat mass (Tsai et al., PLoS One. 2013, 8(2):e55174). An Fc-fusion of GDF15 was shown to decrease body weight and improve glucose tolerance as well as insulin sensitivity in an obese cynomolgus monkey model when administered weekly over a period of six weeks (WO 2013/113008).

The effects of GDF15 on body weight are thought to be mediated via the reduction of food intake and increased energy expenditure. GDF15 improves glycemic control via body weight-dependent and independent mechanisms.

Together, these observations suggest that increasing levels of GDF15 or modulating GDF15 signaling can be beneficial as a therapy for metabolic diseases.

Previous reports have described potential receptors for GDF15 including TGF-beta RII and ALK-5 (Johnen et al., Nat Med. 2007, 13: 1333-1340; Artz et al., Blood 2016, 128:529-41), however these reports lack biochemical evidence showing direct interaction between GDF15 and components of the receptor complex. Therefore the receptor complex and related signaling cascade utilized by GDF15 remains unknown.

There is a need in the art for identification of the cellular targets that mediate the biological effects of GDF15. Identification of GDF15 receptor and downstream signaling targets can aid in developing new treatments and preventive strategies for metabolic diseases, disorders, or conditions.

### SUMMARY OF THE INVENTION

The invention satisfies this need by providing a novel receptor for GDF15, GDNF family receptor alpha like (GFRAL). GFRAL is a distant member of the GDNF family of receptors. The invention demonstrates it's binding to GDF15, the resulting downstream signaling, and in vivo activity.

The invention also provides a method of screening compounds for having GDF15 agonistic activity whereby said compounds have the ability to induce GFRAL-mediated signaling.

The invention also provides a method of screening compounds for having GDF15 antagonistic activity whereby said compounds have the ability to reduce GFRAL-mediated signaling.

In one embodiment, the method comprises the following steps: (a) contacting a cell comprising GFRAL or a fragment thereof with the test compound; (b) contacting a control cell, lacking the expression of GFRAL protein or a fragment thereof, with the test compound; (c) measuring levels of GDF15 biological activity in the test cell and in the control cell; (d) comparing the levels of GDF15 biological activity in the presence of the test compound in the test cell and in the control cell, wherein an increase in the levels of the GDF15 biological activity in the test cell, relative to that in the control cell, indicates that the test compound has GDF15 agonistic activity, and wherein a decrease in the levels of the GDF15 biological activity in the test cell, relative to that in the control cell, indicates that the test compound has GDF15 antagonistic activity. In further embodiments the GDF15 biological activity comprises phosphorylation of tyrosine, phosphorylation of Akt, phosphorylation of Erk1/2, or phosphorylation of PLCγ1. In another embodiment, the method of measuring the levels of the GDF15 biological activity comprises measuring levels of a reporter signal. In another embodiment, the test compound is a part of a library of compounds. In another embodiment, the compound is a composition. In another embodiment, the compound is a fusion protein.

In another embodiment, the method comprises the following steps: (a) contacting a test animal, expressing GFRAL protein, with the test compound; (b) contacting a control animal, lacking the expression of GFRAL protein or a fragment thereof, with the test compound; (c) measuring body weight or food intake in the test animal and the control animal; (d) comparing the body weight or food intake in the presence of the test compound in the test animal and the control animal, wherein the decrease in the body weight or food intake in the test animal relative to that in the control animal, indicates that the test compound has GDF15 agonistic activity; and wherein the increase in the body weight or food intake in the test animal relative to that in the control animal, indicates that the test compound has GDF15 antagonistic activity. In another embodiment, the test compound is a part of a library of compounds. In another embodiment, the compound is a composition. In another embodiment, the compound is a fusion protein.

The invention also provides a kit for screening test compounds for having GDF15 agonistic activity, comprising a cell capable of expressing GFRAL protein and instructions for using the kit in a method for screening test compounds for having GDF15 agonistic activity. In one embodiment, the cell capable of expressing GFRAL protein is a stably or transiently transfected cell.

The invention also provides a kit for screening test compounds for having GDF15 antagonistic activity, comprising a cell capable of expressing GFRAL protein and instructions for using the kit in a method for screening test compounds for having GDF15 antagonistic activity. In one embodiment, the cell capable of expressing GFRAL protein is a stably or transiently transfected cell.

The invention also provides a method of treating a metabolic disorder, comprising administering to a subject a therapeutically effective amount of a compound identified by the method of screening. In one embodiment, the metabolic disorder is selected from the group consisting of type 2 diabetes, hyperglycemia, hyperinsulinemia, obesity, dyslipidemia, diabetic nephropathy, or anorexia.

### DESCRIPTION OF THE FIGURES

FIG. 1 illustrates the binding of the either Fc-GDF15 fusion molecule or Fc alone to GFRAL-overexpressing HEK293F cells, as measured by fluorescence-activated cell sorting (FACS). Grey line represents unstained cells; black line represents Fc control, dotted line represents Fc-GDF15 fusion. % Max Count represents the percentage of the maximal event counts collected by the fluorometer; fluorescent intensity represents the fluorescence of Alexa Fluor 647, measured in relative fluorescence units, using logarithmic scale.

FIG. 2 illustrates the FACS data showing dose-dependent binding curve of Fc-GDF15 fusion molecule to GFRAL-overexpressing HEK293F cells.

FIG. 3 illustrates the dose-dependent binding of HSA-GDF15 ligand to extracellular domain (ECD) of GFRAL. Log ECL signal represents base 10 logarithm of electrochemiluminescence (ECL) signal, measured in arbitrary units.

FIG. 4 illustrates the dose-dependent competition for binding of non-fusion GDF15 and HSA-GDF15 to GFRAL ECD. Log ECL signal represents base 10 logarithm of electrochemiluminescence (ECL) signal, measured in arbitrary units.

FIG. 5 illustrates cell-free assay for binding of either wild type or mutated HSA-GDF15 to GFRAL ECD-Fc, as measured using Meso Scale Discovery platform. Log ECL signal represents base 10 logarithm of electrochemiluminescence (ECL) signal, measured in arbitrary units.

FIG. 6 illustrates the binding of either wild type or mutated HSA-GDF15 to SK-N-AS cells overexpressing GFRAL. Fluorescence, measured in relative fluorescence units, was measured as the geometric mean of three triplicate wells.

FIG. 7 illustrates the effects of GDF15 on protein levels in SK-N-AS cells overexpressing GFRAL.

FIG. 8 illustrates the effects of either wild type of mutant GDF15 on protein levels in SK-N-AS cells overexpressing GFRAL.

FIG. 9 illustrates the effects of GDF15 on protein levels in NG108-15 cells overexpressing GFRAL.

FIG. 10 illustrates levels of gfral expression in mice lacking gfral. Gfral +/+: mice with wild type gfral; gfral +/−: mice heterozygous for gfral deletion; gfral −/−: mice homozygous for gfral deletion.

FIG. 11 illustrates the effects of GDF15 treatment on the amount of food intake over 12 hours in either gfral homozygous knockout mice (B6;129S5-Gfraltm1Lex) or wild type littermate control mice. *: p<0.05 as compared to the wild type mice treated with PBS, using One-Way ANOVA and Tukey tests.

### DETAILED DESCRIPTION OF THE INVENTION

The disclosed subject matter may be understood more readily by reference to the following detailed description taken in connection with the accompanying figures, which form a part of this disclosure. It is to be understood that the disclosed subject matter is not limited to those described and/or shown herein, and that the terminology used herein is for the purpose of describing particular embodiments by way of example only and is not intended to be limiting of the claimed subject matter.

Unless specifically stated otherwise, any description as to a possible mechanism or mode of action or reason for improvement is meant to be illustrative only, and the disclosed subject matter are not to be constrained by the correctness or incorrectness of any such suggested mechanism or mode of action or reason for improvement.

When a range of values is expressed, another embodiment includes from the one particular value and/or to the other particular value. Further, reference to values stated in ranges include each and every value within that range. All ranges are inclusive and may be combined. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed subject matter which are, for clarity, described herein in the context of separate embodiments, may also be provided in combination in a single embodiment. Conversely, various features of the disclosed subject matter that are, for brevity, described in the context of a single embodiment, may also be provided separately or in any subcombination.

### Definitions

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

As used herein, an “agonist” refers to agents which induce activation of receptor signaling pathways, e.g., such as by mimicking a ligand for the receptor, as well as agents which potentiate the sensitivity of the receptor to a ligand, e.g., lower the concentrations of ligand required to induce a particular level of receptor-dependent signaling.

The term “antagonist” refers to agents which either inhibit or decrease activation of receptor signaling pathways.

Broadly speaking, the terms “diabetes” and “diabetic” refer to a progressive disease of carbohydrate metabolism involving inadequate production or utilization of insulin, frequently characterized by hyperglycemia and glycosuria.

“Effective amount” refers to an amount effective, at dosages and for periods of time necessary, to achieve a desired result. An effective amount of a ligand that binds to GFRAL may vary according to factors such as the disease state, age, sex, and weight of the individual, and the ability of the antibody to elicit a desired response in the individual. An effective amount is also one in which any toxic or detrimental effects of the agent are outweighed by the beneficial effects.

As used herein, the term “fusion protein” refers to a protein having two or more portions covalently linked together, where each of the portions is derived from different proteins.

As used herein, “GFRAL” refers to a receptor polypeptide having at least 94% identity to the polypeptide sequence given in SEQ ID NO: 3, and having GFRAL function, or a fragment of the polypeptide sequence given in SEQ ID NO: 3. In some embodiments, said GFRAL has at least 95% identity to the polypeptide sequence given in SEQ ID NO: 3, and having GFRAL function, or a fragment of the polypeptide sequence given in SEQ ID NO: 3. In some embodiments, said GFRAL is the polypeptide sequence given in SEQ ID NO: 3. In other embodiments, said GFRAL is the polypeptide sequence given in SEQ ID NO: 30. In some embodiments said GFRAL is an extracellular domain, such as SEQ ID NO: 19 or SEQ ID NO: 27. GFRAL receptor polypeptides used in the methods of the present invention are preferably mammalian. In some embodiments, the GFRAL receptor polypeptides used in the methods of the present invention are human. In other embodiments, the GFRAL receptor polypeptides used in the methods of the present invention are cynomologous monkey. GFRAL also refers to derivatives of the receptor useful in the screening or rational drug design methods disclosed herein.

The term “hyperglycemia”, as used herein, refers to a condition in which an elevated amount of glucose circulates in the blood plasma of a subject relative to a healthy individual. Hyperglycemia can be diagnosed using methods known in the art, including measurement of fasting blood glucose levels as described herein.

The term “hyperinsulinemia”, as used herein, refers to a condition in which there are elevated levels of circulating insulin when, concomitantly, blood glucose levels are either elevated or normal. Hyperinsulinemia can be caused by insulin resistance which is associated with dyslipidemia, such as high triglycerides, high cholesterol, high low-density lipoprotein (LDL) and low high-density lipoprotein (HDL); high uric acids levels; polycystic ovary syndrome; type II diabetes and obesity. Hyperinsulinemia can be diagnosed as having a plasma insulin level higher than about 2 μU/mL.

A “metabolic disease, disorder or condition” refers to any disorder related to abnormal metabolism. Examples of metabolic diseases, disorders or conditions that can be treated according to a method of the invention include, but are not limited to, type 2 diabetes, elevated glucose levels, elevated insulin levels, obesity, dyslipidemia, or diabetic nephropathy.

“Recombinant” as used herein, includes antibodies and other proteins that are prepared, expressed, created or isolated by recombinant means.

“Subject” refers to human and non-human animals, including all vertebrates, e.g., mammals and non-mammals, such as non-human primates, mice, rabbits, sheep, dogs, cats, horses, cows, chickens, amphibians, and reptiles. In many embodiments of the described subject matter, the subject is a human.

“Treating” or “treatment” refer to any success or indicia of success in the attenuation or amelioration of an injury, pathology, or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters, including the results of a physical examination, neurological examination, or psychiatric evaluations.

### Methods for Screening for Agonists and Antagonists of GDF15 Using GFRAL.

Examples of compounds that can be screened for possessing the properties of either agonist or antagonist of GDF15 include antibodies, antigen-binding proteins, polypeptides, polysaccharides, phospholipids, hormones, prostaglandins, steroids, aromatic compounds, heterocyclic compounds, benzodiazepines, oligomeric N-substituted glycines and oligocarbamates. Large combinatorial libraries of the compounds can be constructed by the encoded synthetic libraries (ESL) method described WO 95/12608, WO 93/06121, WO 94/08051, WO 95/35503, WO 95/30642. Peptide libraries can also be generated by phage display methods. See, e.g., U.S. Pat. No. 5,432,018.

Compounds that can be screened for possessing the properties of either agonist or antagonist of GDF15 include substances which bind to the ligand binding site of GFRAL, substances having an allosteric activity, as well as substances which act non-competitively with respect to the ligand binding site.

Cellular assays generally involve contacting a cell (or more typically a culture of such cells) expressing GFRAL with a test compound and determining whether a property of the cells changes. The change can be assessed from levels of the property before and after contacting the cell with the compound or by performing a control experiment on the control cell or population of cells lacking GFRAL. The property measured may be a level of RNA expression, a level of a protein, a level of modification of a protein, preferably phosphorylation, or a level of a reporter signal.

In one embodiment, an agonist or antagonist of GDF15 may be identified by contacting a cell expressing on the surface thereof the receptor GFRAL, said receptor being associated with a second component capable of providing a detectable signal in response to the binding of a compound to said receptor, with a compound to be screened under conditions to permit binding to the receptor; and determining whether the compound binds to, and activates, or inhibits, the receptor, by detecting the presence or absence of a signal generated from the interaction of the compound with the receptor, optionally in the presence of labeled or unlabeled ligand.

In general, such screening methods involve providing appropriate cells which express GFRAL on the surface thereof. Such cells include cells from mammals (e.g., Chinese hamster ovary (CHO), HEK (human embryonic kidney), SK-N-AS, and cells of Drosophila or E. coli. In particular, a polynucleotide encoding GFRAL is employed to transfect cells to thereby express said receptor. Construction of expression vectors comprising a GFRAL-encoding polynucleotide and transfection of cells with said GFRAL expression vectors can be achieved using standard methods, as described in, for example, Sambrook et al., Molecular Cloning: A Laboratory Manual, 2nd Ed., Cold Spring Harbor Laboratory Press, Cold Spring Harbor, N.Y. (1989). Receptor expression may be transient or stable. Preferably, the expression is stable. More preferably a mammalian cell line is transfected with an expression vector comprising a nucleic acid sequence encoding the GFRAL receptor, for example the polynucleotide of SEQ ID NO: 3, or a fragment or a variant thereof, and the cell line then cultured in a culture medium such that the receptor is stably expressed on the surface of the cell. The expressed receptor is then contacted with a test compound to observe binding, stimulation or inhibition of a functional response, in the presence or absence of a ligand.

Assays as described herein may utilize intact cells expressing functional GFRAL, or cell membranes containing the receptor, as is known in the art.

Alternatively a soluble portion of the GFRAL receptor (i.e. not membrane-bound) comprising the ligand binding domain may be expressed in the soluble fraction, either in the intracellular compartment or secreted out of the cell into the medium. Techniques for the isolation and purification of expressed soluble receptors are well known in the art.

Analogous experiments can be performed on an animal. Suitable biological activities that can be monitored include but are not limited to body weight, food intake, oral glucose tolerance tests, measurements of blood glucose levels, insulin resistance analysis, pharmacokinetic analysis, toxicokinetic analysis, immunoassays and mass spec analysis of the level and stability of full-length fusion proteins, and plasma ex vivo stability analysis.

In another embodiment of this invention, screening assays to identify pharmacologically active ligands for GFRAL are provided. Ligands may encompass numerous chemical classes, though typically they are organic molecules. Such ligands can comprise functional groups necessary for structural interaction with proteins, particularly hydrogen bonding, and typically include at least an amine, carbonyl, hydroxyl or carboxyl group, preferably at least two of the functional chemical groups. Ligands often comprise cyclical carbon or heterocyclic structures and/or aromatic or polyaromatic structures substituted with one or more of the above functional groups. Ligands can also comprise biomolecules including peptides, saccharides, fatty acids, steroids, purines, pyrimidines, derivatives, structural analogs, or combinations thereof.

Ligands may include, for example, 1) peptides such as soluble peptides, including Ig-tailed fusion peptides and members of random peptide libraries (see, e.g., Lam et al., 1991, Nature 354:82-84; Houghten et al., 1991, Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang et al., 1993, Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules.

Ligands can be obtained from a wide variety of sources including libraries of synthetic or natural compounds. Synthetic compound libraries are commercially available from, for example, Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich Chemical Company, Inc. (Milwaukee, Wis.). Natural compound libraries comprising bacterial, fungal, plant or animal extracts are available from, for example, Pan Laboratories (Bothell, Wash.). In addition, numerous means are available for random and directed synthesis of a wide variety of organic compounds and biomolecules, including expression of randomized oligonucleotides.

Alternatively, libraries of natural compounds in the form of bacterial, fungal, plant and animal extracts can be readily produced. Methods for the synthesis of molecular libraries are readily available (see, e.g., DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carell et al., 1994, Angew. Chem. Int. Ed. Engl. 33:2059; Carell et al., 1994, Angew. Chem. Int. Ed. Engl. 33:2061; and in Gallop et al., 1994, J. Med. Chem. 37:1233). In addition, natural or synthetic compound libraries and compounds can be readily modified through conventional chemical, physical and biochemical means (see, e.g., Blondelle et al., 1996, Trends in Biotech. 14:60), and may be used to produce combinatorial libraries. In another approach, previously identified pharmacological agents can be subjected to directed or random chemical modifications, such as acylation, alkylation, esterification, amidification, and the analogs can be screened for GFRAL-modulating activity.

Numerous methods for producing combinatorial libraries are known in the art, including those involving biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide or peptide libraries, while the other four approaches are applicable to polypeptide, peptide, non-peptide oligomer, or small molecule libraries of compounds (K. S. Lam, 1997, Anticancer Drug Des. 12:145).

Libraries may be screened in solution by methods generally known in the art for determining whether ligands bind either competitively or non-competitively at a binding site. Such methods may include screening libraries in solution (e.g., Houghten, 1992, Biotechniques 13:412-421), or on beads (Lam, 1991, Nature 354:82-84), chips (Fodor, 1993, Nature 364:555-556), bacteria or spores (Ladner U.S. Pat. No. 5,223,409), plasmids (Cull et al., 1992, Proc. Natl. Acad. Sci. USA 89:1865-1869), or on phage (Scott and Smith, 1990, Science 249:386-390; Devlin, 1990, Science 249:404-406; Cwirla et al., 1990, Proc. Natl. Acad. Sci. USA 97:6378-6382; Felici, 1991, J. Mol. Biol. 222:301-310; Ladner, supra).

Where the screening assay is a binding assay, GFRAL, or one of the GFRAL-binding ligands, may be joined to a label, where the label can directly or indirectly provide a detectable signal. Various labels include radioisotopes, fluorescent molecules, chemiluminescent molecules, enzymes, specific binding molecules, particles, e.g., magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g., albumin, detergents, etc., which are used to facilitate optimal protein-protein binding and/or reduce non-specific or background interactions. Reagents that improve the efficiency of the assay, such as protease inhibitors, nuclease inhibitors, antimicrobial agents, etc., may be used. The components are added in any order that produces the requisite binding. Incubations are performed at any temperature that facilitates optimal activity, typically between 4° and 40° C. Incubation periods are selected for optimum activity, but may also be optimized to facilitate rapid high-throughput screening. Normally, between 0.1 and 1 hr will be sufficient. In general, a plurality of assay mixtures is run in parallel with different test agent concentrations to obtain a differential response to these concentrations. Typically, one of these concentrations serves as a negative control, i.e., at zero concentration or below the level of detection.

The screening assays provided in accordance with this invention are based on those disclosed in International application WO 96/04557, which is incorporated herein in its entirety. Briefly, WO 96/04557 discloses the use of reporter peptides that bind to active sites on targets and possess agonist or antagonist activity at the target. These reporters are identified from recombinant libraries and are either peptides with random amino acid sequences or variable antibody regions with at least one CDR region that has been randomized. The reporter peptides may be expressed in cell recombinant expression systems, such as for example in E. coli, or by phage display (see WO 96/04557 and Kay et al. 1996, Mol. Divers. 1(2):139-40, both of which are incorporated herein by reference). The reporters identified from the libraries may then be used in accordance with this invention either as therapeutics themselves, or in competition binding assays to screen for other molecules, preferably small, active molecules, which possess similar properties to the reporters and may be developed as drug candidates to provide agonist or antagonist activity. Preferably, these small organic molecules are orally active.

Phage display, yeast display, and mammalian display libraries can also be screened for ligands that bind to GFRAL, as described above. Details of the construction and analyses of these libraries, as well as the basic procedures for biopanning and selection of binders, have been published (see, e.g., WO 96/04557; Mandecki et al., 1997, Display Technologies—Novel Targets and Strategies, P. Guttry (ed), International Business Communications, Inc. Southborogh, Mass., pp. 231-254; Ravera et al., 1998, Oncogene 16:1993-1999; Scott and Smith, 1990, Science 249:386-390); Grihalde et al., 1995, Gene 166:187-195; Chen et al., 1996, Proc. Natl. Acad. Sci. USA 93:1997-2001; Kay et al., 1993, Gene 128:59-65; Carcamo et al., 1998, Proc. Natl. Acad. Sci. USA 95:11146-11151; Hoogenboom, 1997, Trends Biotechnol. 15:62-70; Rader and Barbas, 1997, Curr. Opin. Biotechnol. 8:503-508; all of which are incorporated herein by reference).

The designing of mimetics to a known pharmaceutically active compound is a known approach to the development of pharmaceuticals based on a “lead” compound. This might be desirable where the active compound is difficult or expensive to synthesize or where it is unsuitable for a particular method of administration, e.g., peptides are generally unsuitable active agents for oral compositions as they tend to be quickly degraded by proteases in the alimentary canal. Mimetic design, synthesis, and testing are generally used to avoid large-scale screening of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide (e.g., by substituting each residue in turn). These parts or residues constituting the active region of the compound are known as its “pharmacophore”.

Once the pharmacophore has been found, its structure is modeled according to its physical properties (e.g., stereochemistry, bonding, size, and/or charge), using data from a range of sources (e.g., spectroscopic techniques, X-ray diffraction data, and NMR). Computational analysis, similarity mapping (which models the charge and/or volume of a pharmacophore, rather than the bonding between atoms), and other techniques can be used in this modeling process.

In a variant of this approach, the three dimensional structure of the ligand and its binding partner are modeled. This can be especially useful where the ligand and/or binding partner change conformation on binding, allowing the model to take account of this in the design of the mimetic.

A template molecule is then selected, and chemical groups that mimic the pharmacophore can be grafted onto the template. The template molecule and the chemical groups grafted on to it can conveniently be selected so that the mimetic is easy to synthesize, is likely to be pharmacologically acceptable, does not degrade in vivo, and retains the biological activity of the lead compound. The mimetics found are then screened to ascertain the extent they exhibit the target property, or to what extent they inhibit it. Further optimization or modification can then be carried out to arrive at one or more final mimetics for in vivo or clinical testing.

### Embodiments

Embodiment 1 is a method of screening compounds for having GDF15 agonistic activity whereby said compounds have the ability to induce GFRAL-mediated signaling.

Embodiment 2 is a method of screening compounds for having GDF15 antagonistic activity whereby said compounds have the ability to reduce GFRAL-mediated signaling.

Embodiment 3 is the method according to embodiments 1 or 2, wherein the method comprises the following steps:

(a) contacting a cell comprising GFRAL or a fragment thereof with the test compound;

(b) contacting a control cell, lacking the expression of GFRAL protein or a fragment thereof, with the test compound;

(c) measuring levels of GDF15 biological activity in the test cell and in the control cell;

(d) comparing the levels of GDF15 biological activity in the presence of the test compound in the test cell and in the control cell,

wherein an increase in the levels of the GDF15 biological activity in the test cell, relative to that in the control cell, indicates that the test compound has GDF15 agonistic activity,

and wherein a decrease in the levels of the GDF15 biological activity in the test cell, relative to that in the control cell, indicates that the test compound has GDF15 antagonistic activity.

Embodiment 4 is the method according to embodiments 1 or 2, wherein the method comprises the following steps:

(a) contacting a test animal, expressing GFRAL protein, with the test compound;

(b) contacting a control animal, lacking the expression of GFRAL protein or a fragment thereof, with the test compound;

(c) measuring body weight or food intake in the test animal and the control animal;

(d) comparing the body weight or food intake in the presence of the test compound in the test animal and the control animal,

wherein the decrease in the body weight or food intake in the test animal relative to that in the control animal, indicates that the test compound has GDF15 agonistic activity;

and wherein the increase in the body weight or food intake in the test animal relative to that in the control animal, indicates that the test compound has GDF15 antagonistic activity.

Embodiment 5 is the method according to embodiment 3 wherein the GDF15 biological activity comprises phosphorylation of tyrosine.

Embodiment 6 is the method according to embodiment 3 wherein the GDF15 biological activity comprise phosphorylation of Akt.

Embodiment 7 is the method according to embodiment 3 wherein the GDF15 biological activity comprise phosphorylation of Erk1/2.

Embodiment 8 is the method according to embodiment 3 wherein the GDF15 biological activity comprise phosphorylation of PLCγ1.

Embodiment 9 is the method according to embodiment 3 wherein measuring the levels of the GDF15 biological activity comprise measuring levels of a reporter signal.

Embodiment 10 is the method according to embodiments 3 or 4 wherein the compound is a part of a library of compounds.

Embodiment 11 is the method according to embodiments 3 or 4 wherein the compound is a composition.

Embodiment 12 is the method according to embodiments 3 or 4 wherein the compound is a fusion protein.

Embodiment 13 is the method of claim 3 or 4 wherein GFRAL comprises a sequence having at least 94% identity to human GFRAL extracellular domain sequence.

Embodiment 14 is a kit for screening test compounds for having GDF15 agonistic activity, comprising a cell capable of expressing GFRAL protein and instructions for using the kit in a method for screening test compounds for having GDF15 agonistic activity.

Embodiment 15 is the kit according to embodiment 14, wherein the cell capable of expressing GFRAL protein is a stably or transiently transfected cell.

Embodiment 16 is a kit for screening test compounds for having GDF15 antagonistic activity, comprising a cell capable of expressing GFRAL protein and instructions for using the kit in a method for screening test compounds for having GDF15 antagonistic activity.

Embodiment 17 is the kit according to embodiment 16, wherein the cell capable of expressing GFRAL protein is a stably or transiently transfected cell.

Embodiment 18 is a method of treating a metabolic disorder, comprising administering to a subject a therapeutically effective amount of a compound identified by the method of embodiments 1 or 2.

Embodiment 19 is the method of claim 18 wherein the metabolic disorder is selected from the group consisting of type 2 diabetes, hyperglycemia, hyperinsulinemia, obesity, dyslipidemia, diabetic nephropathy, or anorexia.

The following examples illustrate the invention. These examples should not be construed as to limit the scope of this invention. The examples are included for purposes of illustration and the present invention is limited only by the claims.

### Example 1. Identification of GDF15 Binding Partners

Two approaches of screening DNA libraries were used to identify a receptor for GDF15. The Janssen internal library, consisting of cDNA encoding 3048 cell surface receptors, was used for cell surface expression and screening for binding partners to a heterodimeric Fc-GDF15 fusion molecule, consisting of a Fc-GDF15 fusion chain (SEQ ID NO: 1) dimerized with a Fc alone chain (SEQ ID NO: 2), using ImageXpress High Content Imaging System (Molecular Devices). For transfecting DNA of Janssen membrane library, HEK293F cells were plated at the density of 30,000 cells per well in growth media (100 μl DMEM, 10% FBS and 250 μg/ml Geneticin, all three reagents from Thermo Fisher Scientific) onto clear bottom 96-well plates (Perkin Elmer). The following day, 100 ng of DNA premixed with Lipofectamine 2000 (Thermo Fisher Scientific) was added to each well of the cell plates. After 24 hours, media was aspirated and 50 μl of detection reagents containing 2 μg/ml Fc-GDF15 ligand (Janssen), 2 μg/ml R-Phycoerythrin labeled anti-human Fc antibody (Jackson Immuno Research) and 10 μM Hoechst (Jackson Immuno Research) was added to each well. Plates were incubated for at least 3 hours in 4° C. before they were imaged on ImageXpress (Molecular Devices) using appropriated filter channels. For the primary screen, each plate had wells transfected with FcγR1A as positive control for transfection and binding, as well as wells of non-transfected cells as a control for background binding signal. Each well was imaged with four fields of view. Images were evaluated by visual inspection to determine if there is any binding. The primary hits were scaled up and sequence confirmed for confirmation screening. The confirmation screening was carried out with the same protocol as the primary screen, with the addition of another testing ligand HisTagged-HSA-GDF15 (Janssen) and two negative control ligands: Fc molecule (Janssen) and HisTagged-HSA (Janssen) molecule to assess the specificity of the hits. The detection of the HSA fusions were done through the binding of the HisTag and the mouse anti-His antibody (Genscript) as well as and R-Phycoerythrin labeled anti-mouse antibody (Jackson Immuno Research), both at 2 μg/ml in the detection reagent.

Out of the 3048 receptors, primary screening identified 41 hits that bound to the Fc-GDF15 ligand, among which 6 were Fc receptors. The remaining 35 hits were tested in confirmation screening and non-reproducible and non-specific hits were filtered out. Only one hit, Neuropilin 2 (NRP2), was confirmed in the secondary screens to bind to Fc-GDF15 ligand, but not to Fc molecule alone.

In parallel, two studies were performed at Retrogenix Ltd (Whaley Bridge, High Peak, Derbyshire, UK) using Retrogenix' Cell Microarray technology to screen for binding partners for the Fc-GDF15 fusion molecule. Two studies were performed to screen Retrogenix's plasma membrane protein library, first on 3500 proteins and second on an additional 993 proteins, with total number of proteins screened being 4493. A background screen was performed prior to the primary screen to detect the background level of binding of the test ligand Fc-GDF15 at 2, 5, and 20 ug/ml with blank slides coated with live HEK293 cells and detected by using an AlexaFluor647 anti-Fc antibody. In the primary screen, vectors encoding each full-length human plasma membrane protein in the library were arrayed in duplicates on Retrogenix's cell microarray slides (referred to as ‘slide-set’). Three replicate slide-set were used in the primary screen. Control expression vector (pIRES-hEGFR-IRES-ZsGreen1) was spotted in quadruplicate on every slide to ensure that a minimal threshold of transfection efficiency had been achieved or exceeded on every slide. HEK293 cells were used for reverse transfection in live conditions. The test ligand Fc-GDF15 was added at the concentration of 20 ug/ml to each slide set. After the addition of test ligand, cell fixation was performed and the AlexaFluor647 anti-Fc antibody was used for binding detection. Fluorescent images were analyzed using ImageQuant software (GE) and a protein ‘hit’ is defined as a duplicate spot showing a raised signal compared to background levels by visual inspection using the images gridded on the ImageQuant software. Hits identified in the primary screen were tested in a confirmation screen, where all vectors encoding the hits in at least one of the three replicate primary screening slide-sets were arrayed on new slides. Confirmation screening was performed similar to the primary screening, with the addition of control samples for binding Fc. Binding of cells overexpressing CD86 to human CTLA4 fused to human Fc, served as a positive control, and binding to Fc alone served as a negative control.

In the first study that included 3500 proteins, 59 hits were identified from the primary screen with low stringency on intensity for classifying ‘hits’. These 59 primary hits were further investigated in the confirmation screening, which showed that 31 of the hits were not reproducible and 15 of the primary hits were non-specific as indicated by interaction with at least one negative control. The remaining 13 hits were reproducible and specific, with classification as either very weak or weak hits. In the second study that included an additional 993 proteins, 10 primary hits were identified, with 9 of them found to be non-specific in the confirmation screen and 1 remaining hit to be reproducible and specific, with weak/medium binding intensity.

All the binding hits identified from Janssen and Retrogenix libraries are listed in Table 1. They were further investigated for being true binders. Specifically, the binding of NRP2 to Fc-GDF15 ligand did not reproduce when COL0829 cells were used, a cell line that endogenously expressed NRP2 (data not shown). The hits identified by the confirmation of the Retrogenix screen were retested for binding to Fc-GDF15 ligand. In addition, these hits were carefully examined for potential biological relevance with GDF15. GFRAL, a previously little-studied orphan receptor, was identified in the Retrogenix screen (Table 1). It is closely related to GDNF-receptor family (Li et al., Journal of Neurochemistry 2005; 361-376). Furthermore, the ligands for the GDNF family and GDF15 belong to the same family of TFGβ and have structural homology (Shi et al., Nature 2011; 474, 343-349). Thus the binding of GFRAL to GDF15 was thoroughly investigated.

TABLE 1
GDF15 binding hits from library screens.

| Library | Gene ID | Description |
|---|---|---|
| Janssen | NRP2 | Neuropilin 2 |
| Retrogenix | PIGR | Polymeric Immunoglobulin Receptor |
| | TMED1 | Transmembrane emp24 protein transport domain |
| | | containing 1 |
| | BAI1 | Brain-specific angiogenesis inhibitor 1 |
| | FCRL5 | Fc receptor-like 5 |
| | GFRAL | GDNF Family Receptor Alpha Like |

### Example 2. Confirmation of GDF15 Binding Partners Using Cells Overexpressing GFRAL

Confirmation of binding was repeated using in-house designed GFRAL expression constructs followed by FACS analysis. The DNA of full-length GFRAL, side-by-side with the closest members in the GDNF receptor family was transiently transfected into HEK293F cells.

Design of Expression Constructs.

The expression constructs for GFRAL and GFRα family members were made using a pUnder based expression vector, driven by a CMV promoter. The coding region of the constructs was composed of a recombinant signal peptide known to drive strong protein expression and secretion (SEQ ID NO:11), a flag tag (SEQ ID NO:12) and the full length protein, leaving out the predicted endogenous signal peptide, of GFRAL (SEQ ID NO:13), GFRα1 (SEQ ID NO:14), GFRα2 (SEQ ID NO:15), GFRα3 (SEQ ID NO:16) and GFRα4 (SEQ ID NO:17). A Kozak sequence (SEQ ID NO:18) was placed in front of the start codon. The coding regions were codon optimized for mammalian expression and constructs were made by gene synthesis and molecular cloning. The same pUnder-based vector was used to make GFRAL-ECD constructs. The predicted extra cellular domain (ECD) of GFRAL (SEQ ID NO:19) was preceded by the recombinant signal peptide described previously, and followed by the C-terminal protein tags. Two construct designs were made, one with 6×His-tag and an Avi-tag (SEQ ID NO:20) at the C-terminus and the other one with the human IgG1 Fc, 6×His-tag and an Avi-tag at the C-terminus (GFRAL ECD-Fc). The coding regions were codon optimized for mammalian expression and constructs were made using standard gene synthesis and molecular cloning methods.

The GFRAL ECD proteins were expressed in Expi293™ cells by transient transfection using ExpiFectamine™ 293 transfection kit according to the manufacturer's protocol, and were purified by immobilized metal ion affinity chromatography (IMAC) followed by size-exclusion chromatography (SEC). Briefly, for each protein, the clarified cell supernatant was applied to a HisTrap HP column, followed by a stepwise elution with increasing imidazole concentration (10-500 mM). Fractions containing GFRAL ECD were identified by SDS-PAGE and pooled. The protein was filtered using a 0.2 μm membrane and concentrated to an appropriate volume before loading onto a HiLoad 26/60 Superdex 200 pg column (GE Healthcare) equilibrated with 1×DPBS, pH 7.2. Protein fractions eluted from the SEC column with high purity (determined by SDS-PAGE) were pooled and stored at 4° C. Protein concentration was determined by absorbance at 280 nm on a NanoDrop® spectrophotometer (Thermo Fisher Scientific). The quality of the purified proteins was assessed by SDS-PAGE and analytical size exclusion HPLC (Tosoh TSKgel BioAssist G3SW_{XL}). Endotoxin levels were measured using an LAL assay (Associates of Cape Cod, Inc.). Purified proteins were stored at 4° C. in 1×DPBS, pH 7.2.

Transient Transfections.

Free Style™ 293-F cells (HEK293F, Invitrogen) were transfected using 293fectin Transfection Reagent (Invitrogen) following the manufacturer's protocol. Briefly, the DNA/293fectin mixture was made by adding 3 μl of DNA at 100 ng/μl to 17 μl of diluted 293fectin (35 μl of 293fectin to 1 ml OptiMEM. The resulting DNA/293fectin mixture containing 300 ng DNA and 0.6 μl 293fectin in a total volume of 20 μl was incubated at RT for 20-30 min. 200 μl of 2e6 cells/ml then was added to the DNA/293fectin mixture, mixed, and then transferred to a deep well plate which was covered and shaken in a CO₂ incubator at 745 rpm for 2 days. Cells were harvested and subjected to FACS staining two days post transfection.

FACS Binding Experiments.

For the confirmation of specific binding of GDF15 to GFRAL, HEK293F cells expressing the N-terminally flag tagged GFRAL, GFRα1, GFRα2, GFRα3 or GFRα4 were incubated with Fc-GDF15 and the binding was analyzed by FACS. Briefly, transiently transfected cells were spun down two days post transfection, washed with 1×BD staining buffer (BD Pharmingen) and treated with 5 μg/ml of Fc-GDF15 by incubating at 4° C. for 1 hour in 1×BD staining buffer (BD Pharmingen). Cells were subsequently washed and stained with the secondary antibody, goat anti-human IgG conjugated with Alexa Fluor 647 (AF647, Life Technologies), at 4° C. for 30 min, for the detection of Fc-GDF15 binding. Cells from the same batch of transfection were also stained with an Fc isotype negative control, and with FITC-labeled anti-flag antibody (Sigma) for the detection of cell surface expression, and DAPI for live/dead staining. The stained cells were analyzed on BD Fortessa LSR.

Design, Expression, and Purification of GDF15, HSA-GDF15 and Fc-GDF15.

GDF15 homodimer was designed as the full length protein (SEQ ID NO:21), with an EcoRI site and a FLAG tag (SEQ ID NO:22) inserted after the native furin cleavage site between Arg196-Ala197, as previously described (Bauskin A. R. et al. (2000) EMBO J. 19(10): 2212-20). This expression gene was inserted into a mammalian expression vector under the control of a CMV promoter. To generate the mature GDF15 homodimer, the full length protein was co-expressed transiently in Expi293™ (Thermo Fisher Scientific) cells with a plasmid encoding furin protease (Janssen) for intracellular processing using ExpiFectamine™ 293 transfection kit (Thermo Fisher Scientific) according to the manufacturer's protocol. Secreted mature GDF15 homodimer was isolated from the clarified cell supernatant by batch binding to Anti-FLAG® M2 affinity resin (Sigma Aldrich) for 16-24 h at 4° C., followed by elution with 0.1 M glycine, pH 3.5. Fractions identified by SDS-PAGE to contain the protein of interest were pooled and dialyzed against 1×DPBS (Dulbecco's phosphate-buffered saline), pH 7.2 and stored at 4° C.

HSA-GDF15 was designed as HSA (SEQ ID NO:23) fused to the N-terminus of mature GDF15 (AA 197-308) via a linker (SEQ ID NO:24). In addition, an EcoRI site and a 6×His fusion were added to the N-terminus of HSA to facilitate cloning and purification, respectively. The final gene was inserted into a mammalian expression vector with a murine Ig heavy chain secretion tag and under the control of a CMV promoter. HSA-GDF15 homodimer was expressed in Expi293™ cells by transient transfection using ExpiFectamine™ 293 transfection kit according to the manufacturer's protocol, and purified by immobilized metal ion affinity chromatography (IMAC) followed by size-exclusion chromatography (SEC). Briefly, the clarified cell supernatant was applied to a HisTrap HP column, followed by a stepwise elution with increasing imidazole concentration (10-500 mM). Fractions containing HSA-GDF15 dimer were identified by SDS-PAGE and pooled. The protein was filtered using a 0.2 μm membrane and concentrated to an appropriate volume before loading onto a HiLoad 26/60 Superdex 200 pg column (GE Healthcare) equilibrated with 1×DPBS, pH 7.2. Protein fractions eluted from the SEC column with high purity (determined by SDS-PAGE) were pooled and stored at 4° C.

To generate Fc-GDF15 a ‘knob-in-hole’ strategy was utilized, where the ‘knob’ Fc has a T366W mutation and the ‘hole’ Fc has T366S/L368A/Y407V mutations for preferential heterodimer formation. Specifically, GDF15 (AA 197-308) was fused to the C-terminus of human IgG4 Fc with ‘hole’ mutations through a linker (SEQ ID NO:25) (‘hole’ Fc-GDF15). The complementary human IgG4 with ‘knob’ mutations (‘knob’ Fc) was designed without a fusion partner, which, when combined with ‘hole’ Fc-GDF15, should ultimately form a GDF15 homodimer with an Fc knob-in-hole heterodimer fusion at each N-terminus. The two expression genes, ‘hole’ Fc-GDF15 and ‘knob’ Fc, where inserted into separate mammalian expression vectors, each with a murine Ig heavy chain secretion tag and under the control of a CMV promoter. Protein was expressed in Expi293™ cells by transient transfection using ExpiFectamine™ 293 transfection kit according to the manufacturer's protocol, and purified using Protein A affinity column followed by size-exclusion chromatography (SEC). Briefly, the clarified cell supernatant was applied to a HiTrap MabSelect SuRe column (GE Healthcare), followed by elution with 0.1 M Na-acetate, pH 3.5. Fractions containing Fc-GDF15 dimer were identified by SDS-PAGE and pooled. The protein was filtered using a 0.2 μm membrane and concentrated to an appropriate volume before loading onto a HiLoad 26/60 Superdex 200 pg column equilibrated with 1×DPBS, pH 7.2. Protein fractions eluted from the SEC column with high purity (determined by SDS-PAGE) were pooled and stored at 4° C.

The concentrations of all proteins were determined by absorbance at 280 nm on a NanoDrop® spectrophotometer (Thermo Fisher Scientific). The quality of the purified proteins was assessed by SDS-PAGE and analytical size exclusion HPLC (Tosoh TSKgel BioAssist G3SW_{XL}). Endotoxin levels were measured using an LAL assay (Associates of Cape Cod, Inc.). All purified proteins were stored at 4° C. in 1×DPBS, pH 7.2.

Results.

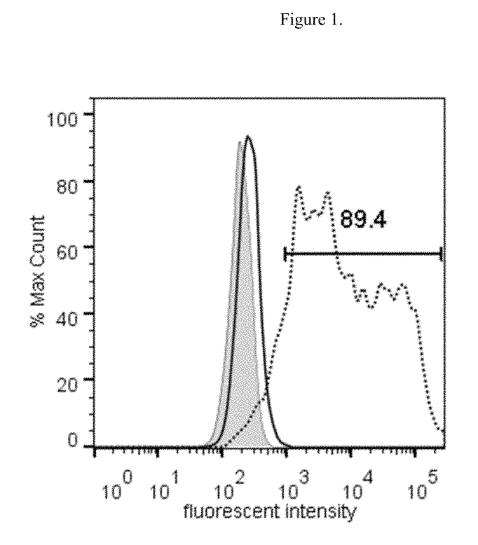

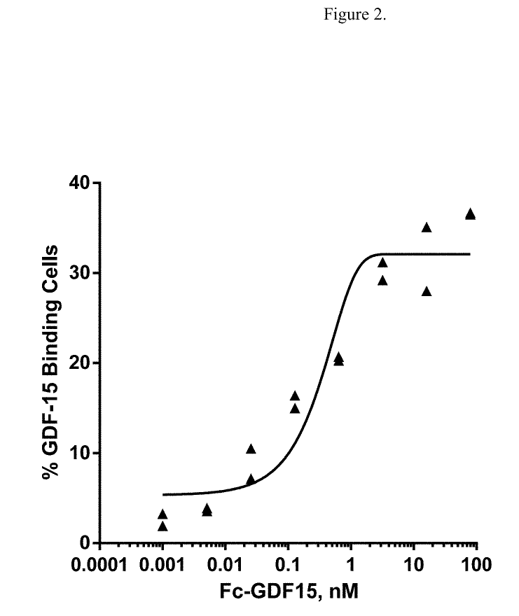

The results demonstrated that only GFRAL-expressing cells bound to Fc-GDF15 ligand, but not to Fc molecule alone (FIG. 1). Cells transfected with other GDNF family members (GFRα1-4) did not bind to Fc-GDF15 (data not shown), indicating that the binding of GDF15 is specific to GFRAL. In addition, receptors GFRα1-4 were included in the Retrogenix library and there was no detected binding of GDF15 to these receptors in the initial screen. The binding of Fc-GDF15 to GFRAL-expressing cells was dose-dependent with an EC50=0.2577 nM (FIG. 2).

### Example 3. Confirmation of GDF15 Binding Partners Using Cells-Free System

The protein of GFRAL extracellular domain (ECD)-Fc fusion was made recombinantly and tested for binding to an HSA-GDF15 ligand in a cell-free plate-based format.

Meso Scale Discovery Binding Assay

A plate-based assay was developed for testing the GDF15-GFRAL ECD binding in a cell-free system. Briefly, GFRAL ECD-Fc molecule was coated on MSD standard plates (Meso Scale Discovery) overnight at 4° C. at 4 μg/ml in PBS. The next day, the plates were washed 3 times in PBS with 0.05% Tween 20 and blocked for 30 minutes by StartingBlock blocking buffer (Thermo Fisher Scientific). For binding experiments, HSA-GDF15 ligand at concentrations ranging from 0.02 pM to 100 nM was added to the plates at 25 μl per well and incubated for 1 hour. For competition experiments of non-fusion GDF15 with HSA-GDF15 ligands, fixed concentration of HSA-GDF15 at 6.25 nM was premixed with different concentrations of non-fusion GDF15 starting from 100 nM for 30 minutes before 25 μl of the mixture was added to each well. After another three washes with PBS-tween, 25 μl of detection regent containing 1 μg/ml mouse anti-HSA antibody (Kerafast Inc) and 1 μg/ml SulfoTag anti-mouse antibody was added to each well and incubated for another hour. The plates were then washed three times with PBS-Tween and 150 μl of read buffer (Meso Scale Discovery) was added before it was read in plate reader (Meso Scale Discovery Sector instrument). Each condition was tested in duplicate wells and the average was plotted and analyzed in GraphPad Prism software. Four-parameter least squares fit was performed to the dose-response curves.

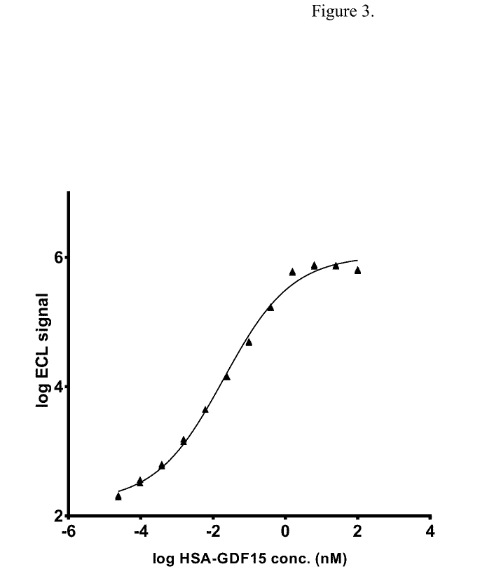

The dose-dependent curve of the HSA-GDF15 binding to GFRAL ECD-Fc also showed a sub-nM EC50 (0.02 nM) (FIG. 3) in this binding format.

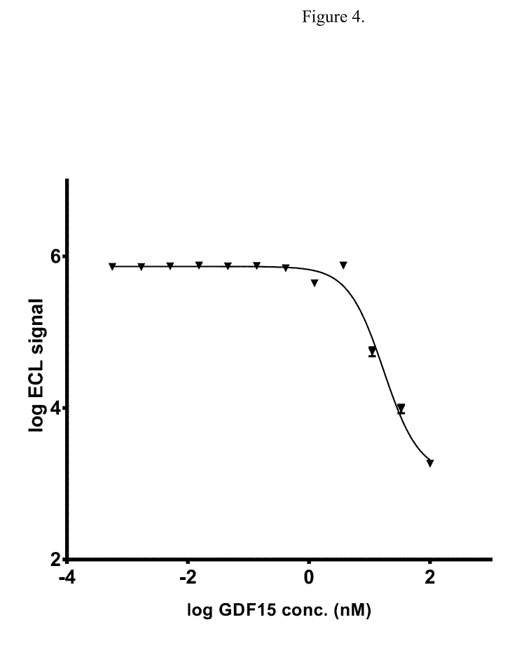

Next, competition assay was performed using fixed concentration of HSA-GDF15 at 6.25 nM, and a range of concentration of non-fusion GDF15 up to 100 nM. The result showed clear competition, indicated by the decrease in signal, when the non-fusion GDF15 concentration is at or greater than the HSA-GDF15 concentration of 6.25 nM (FIG. 4). Finally, surface plasmon resonance (SPR) measurement was performed between GFRAL ECD-Fc and both GDF15 and HSA-GDF15 ligands. The measured affinity from three independent experiments with 4 replicates showed that GDF15 has KD of 19.6±5.08 pM and HSA-GDF15 has KD of 318±69 pM (Table 2). The 16-fold difference in KD between the non-fusion and HSA-fusion GDF15 is predominantly contributed by a much faster ka than a slower kd, suggesting the HSA fusion molecule might cause some steric hindrance to the GFRAL receptor.

TABLE 2
Binding kinetics for GDF15 and HSA-GDF15 as measured by SPR.

| Ligand | ka (1/Ms) 10⁶ | kd (1/s) 10⁻⁰⁴ | KD (pM) |
|---|---|---|---|
| FlagTag-GDF15, | 10.1 ± 1.43 | 1.97 ± 0.49 | 19.6 ± 5.08 |
| homodimer | | | |
| HisTag-HSA-GDF15, | 1.41 ± 0.17 | 4.44 ± 0.85 | 318 ± 69 |
| homodimer | | | |

### Example 4. Binding of Biologically Inactive Mutants to GFRAL

Because GRFAL belongs to GDNF receptor family and the closest family members GFRα1-4 all have RET as a co-receptor, RET was studied for a potential co-receptor for GFRAL binding to GDF15. Structure based on homology model of GDF15:GFRAL:RET was examined for the potential epitopes on GDF15, GFRAL and RET that would interact (data not shown). According to the model, a surface on GDF15 approximating that employed by some TGF-beta superfamily members in their engagement of canonical Type II receptors is likely to interact with the D2 domain of the GFRAL ECD. Interactions between GFRAL and RET are likely to be distributed across multiple domains.

Several point mutants of surface amino acids of GDF15 on the HSA fusion platform were designed and generated with the purpose of eliminating GDF15 biological activity for the investigation of receptor-interacting epitopes on GDF15 (see application Ser. No. 62/333,886). The molecular integrity of these mutants was examined by SDS-PAGE, HPLC, and binding to anti-GDF15 antibodies. The similar size bands in SDS-PAGE, similar retention time and peak shape by HPLC and similar binding curves to multiple anti-GDF15 antibodies compared with wild type GDF15 (application Ser. No. 62/333,886) suggested that the overall molecular conformation is not substantially interrupted by the point mutations.

The HSA-GDF15 point mutants, one biologically active (Q60W) and two biologically inactive (I89R and W32A), were then tested for binding to GFRAL ECD-Fc in the cell-free plate-based binding assay. Briefly, GFRAL ECD-Fc molecule was coated on MSD standard plates (Meso Scale Discovery) overnight at 4° C. at 4 μg/ml in PBS. The next day, the plates were washed 3 times in PBS with 0.05% Tween 20 and blocked for 30 minutes by StartingBlock blocking buffer (Thermo Fisher Scientific). HSA-GDF15 ligand and its mutants at different concentrations were added to the plates at 25 μl per well and incubated for 1 hour. After another three washes with PBS-tween, 25 μl of detection regent containing 1 μg/ml mouse anti-HSA antibody (Kerafast Inc) and 1 μg/ml SulfoTag anti-mouse antibody was added to each well and incubated for another hour. The plates were then washed three times with PBS-Tween and 150 μl of read buffer (Meso Scale Discovery) was added before it was read in the plate reader (Meso Scale Discovery Sector instrument). Each condition had a duplicated well and the average was plotted and analyzed in GraphPad Prism software. Four-parameter least squares fit was performed to the dose-response curves.

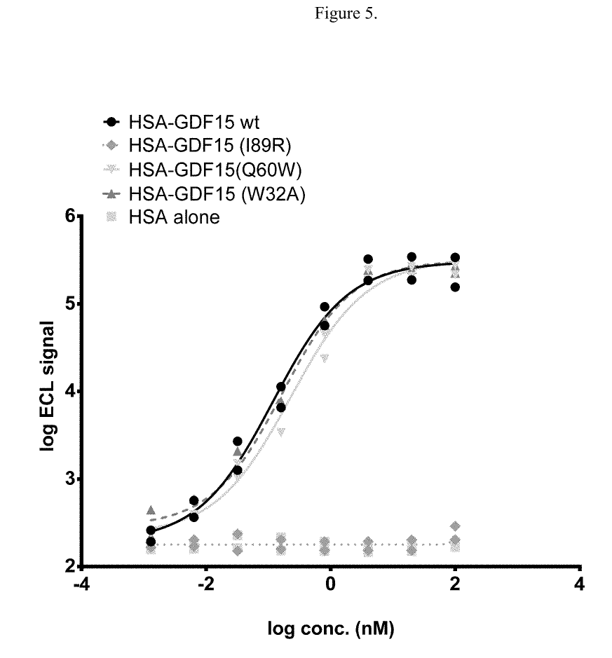

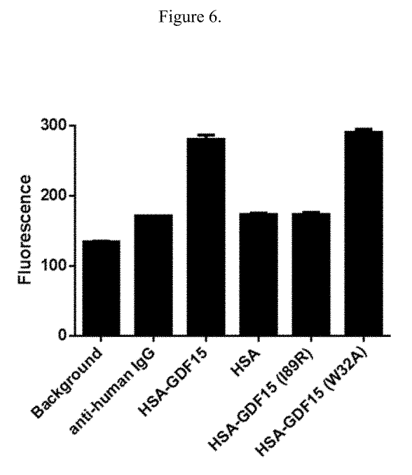

Results showed that while the biologically active mutant (Q60W) had similar binding profile as the wild type HSA-GDF15, the two biologically inactive mutants differed in their binding: The I89R mutant completely lost binding to GFRAL ECD-Fc while the W32A mutant has overlapping binding curve as the wild type (FIG. 5). The binding of these point mutants were also characterized in cells transfected with full-length GFRAL. The binding results by FACS on the GFRAL-expressing cells are consistent with that of the plate-based ECD: Out of the two biologically inactive mutants, only the W32A mutant, but not the I89R mutant, has similar geometric mean of florescence intensity compared to the wild type (FIG. 6).

The finding that both the I89R and W32A mutants were biologically inactive while only the I89R mutant lost binding to GFRAL is consistent with the structural model. Whereas the in silico analysis suggests the I89 residue is in the GFRAL-interacting epitope, it also indicates a RET co-receptor may plausibly interact with GDF15 around the W32 residue. This is consistent with experimental data that while W32 mutant still binds to GFRAL, it does not have biological activity in vivo due to lack of co-receptor interaction.

### Example 5. GDF15-Induced In Vitro Signaling in GFRAL-Overexpressing Cell

GDNF family ligands bind specific GFRα receptors and signal through the activation of the RET receptor tyrosine kinase. Upon activation, RET is phosphorylated on multiple tyrosine residues which triggers multiple signaling pathways, including phosphorylation of serine/threonine kinase Akt, a mitogen-activated protein kinase Erk1/2, and a phosphoinositide-specific phospholipase Cγ1 (PLCγ1) (reviewed in Mulligan, Nature Reviews Cancer 2014, 14, p. 173-186). Thus the activation of RET mediated signaling pathways through the binding of GDF15 to GFRAL was thoroughly investigated.

SK-N-AS and NG108-15 cells overexpressing GFRAL were used to investigate the signaling pathway of GDF15 stimulation. SK-N-AS cells were maintained in Dulbecco's Modified Eagle's Medium (DMEM) supplemented with 10% fetal bovine serum (FBS) and 0.1 mM Non-Essential Amino Acids (NEAA). NG108-15 is a hybrid mouse cell line of neuroblastoma (N18TG-2) and rat glioma (C6BU-1). NG108-15 cells were maintained in DMEM with 4.5 g/L glucose and supplemented with 10% FBS, 10 mM hypoxanthine, 0.1 mM aminopterin, and 1.6 mM thymidine. For transient transfections, cells were plated in 6-well plates at 30% confluence and incubated until cells reached ˜75-80% confluence. Transient transfections of SK-N-AS and NG108-15 cells were conducted using Lipofectamine 2000 in Opti-MEM according to the manufacturer's recommendations. Cells were cultured in 6-well plates and the DNA-to-Lipofectamine 2000 ratio used was 4 μg DNA:10 μl Lipofectamine 2000. Phospho-signaling assessment was performed approximately 40 hours after transfection.

To assay the expression level of a protein, or the phosphorylation state of a signaling protein, Western blots were performed. For immunoblot analysis, cells were serum-starved for 16 hr and stimulated for 15 min with recombinant NRTN or GDF15 at 50 ng/ml unless otherwise stated. After stimulation the cells were washed twice with ice cold PBS before 90 μL Cell Signaling lysis buffer (Cell Signaling Technologies) was added. Cells were scraped off the dish using a cell scraper and transferred to an Eppendorf tube and kept on ice for 20 minutes. The samples were sonicated 3 times for 10 seconds and then centrifuged at 16,000×g for 10 minutes at 4° C. Supernatants were transferred to a fresh tube and stored at −80° C.

The protein concentration was determined by Bicinchoninic acid (BCA) Protein Assay Kit (from Pierce). Sample buffer was added to the lysates and heated at 95° C. for 5 minutes and then cooled down. Equal amounts of proteins were loaded onto NuPAGE 4-12% Bis-Tris gels (Invitrogen), and run for 50 minutes at 150 Volts in NuPAGE MES-SDS running buffer and subsequently transferred to nitrocellulose membranes using the iBlot transfer system (ThermoFisher). Membranes were blocked for 60 minutes using LI-COR blocking buffer. Primary antibody incubations were performed at different optimal dilutions for different antibodies. All incubations for primary antibodies were performed overnight at 4° C. followed by secondary antibody (Alexa Fluor 680 or Alexa Fluor 800 from LI-COR) incubation at 1:10000 dilutions for 1 hour at room temperature. After 3× washing, membranes were scanned with the Odyssey Infrared Imaging System (LI-COR).

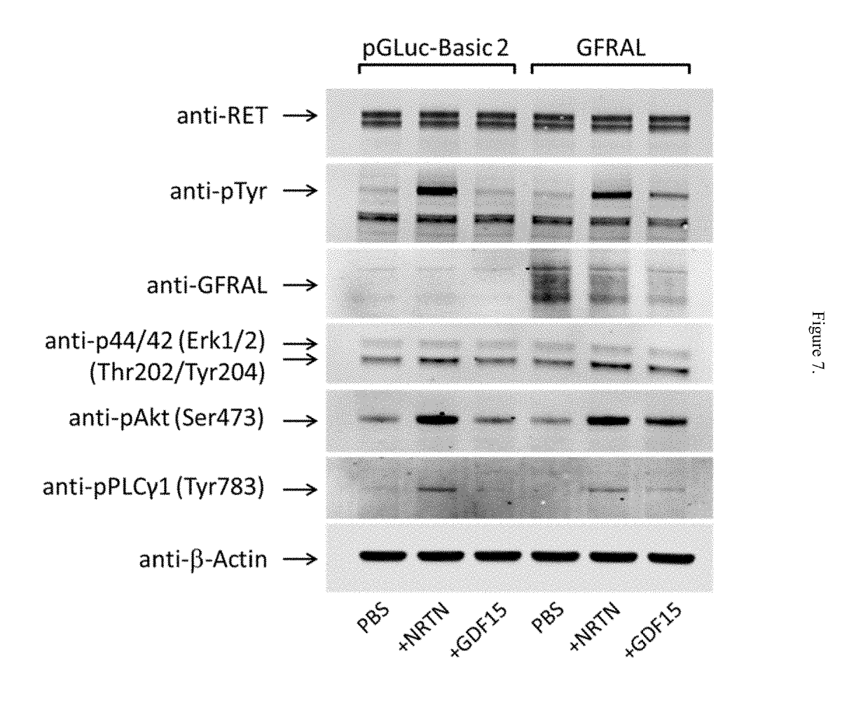

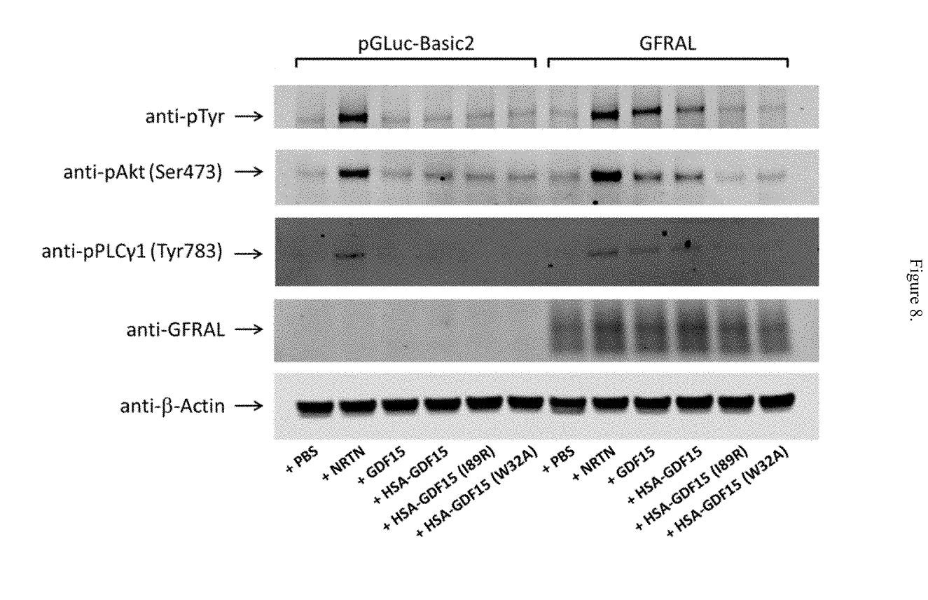

SK-N-AS cells endogenously express GFRα2 and RET, the native receptors for another GDNF family ligand Neurturin (NRTN). Without GFRAL transfection (FIG. 7, left half), adding NRTN, but not GDF15 induces stronger band in phospho-Tyr, phospho-Akt, phospho-Erk1/2 and phospho-PLCγ1. When GFRAL is transfected (FIG. 7, right half), non-fusion GDF15 addition also induces signaling in phospho-Tyr, phospho-Akt, phospho-Erk1/2 and phospho-PLCγ1 (FIG. 7, lane 6 compared with lane 4). The phospho-signaling was confirmed with the HSA-GDF15 molecule (FIG. 8, lane 10). However, the HSA fusion of two biologically-inactive GDF15 point mutants at positions W32 and I89 failed to elicit the signaling on phospho-Tyr, phospho-Akt, phospho-Erk1/2, and phospho-PLCγ1 in SK-N-AS cells (FIG. 8, lane 11 and 12).

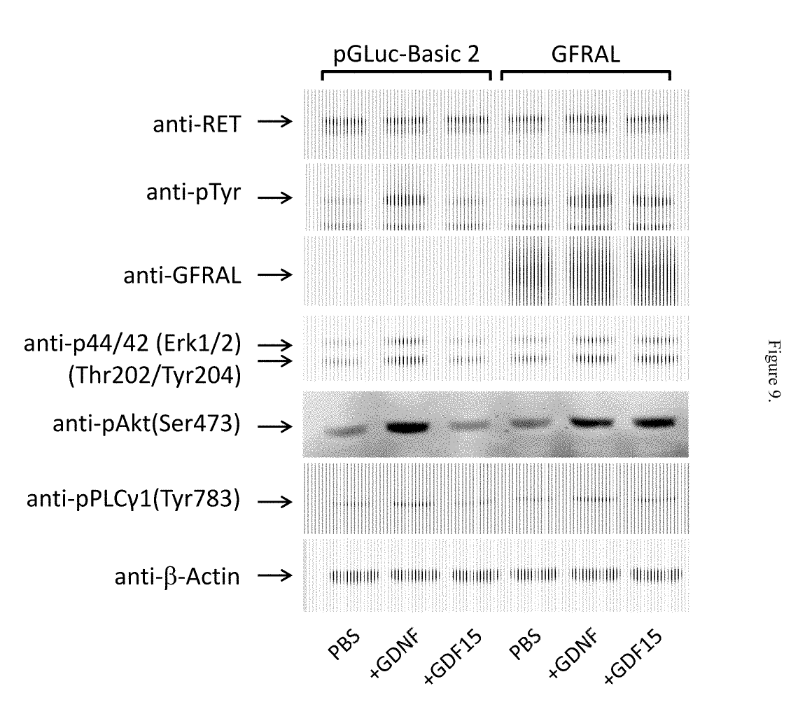

NG108-15 cells endogenously express GFRα1 and RET, the native receptors for ligand GDNF. Without GFRAL transfection (FIG. 9, left half), adding GDNF, but not GDF15 induces stronger band in phospho-Tyr, phospho-Akt, phospho-Erk1/2, and phospho-PLCγ1. When GFRAL is transfected (FIG. 9, right half), non-fusion GDF15 addition also induces signaling in phospho-Tyr, phospho-Akt, phospho-Erk1/2, and phospho-PLCγ1 (FIG. 9, lane 6 compared with lane 4).

### Example 6. GFRAL Receptor Mediates GDF15 Effects In Vivo

Cohorts of B6;129S5-Gfraltm1Lex (Gfral−/−) constitutive knock out (KO) mice, as well as wild type littermate control (Gfral+/+) mice, were obtained from a breeding colony stemming from Taconic Biosciences Model TF3754 and maintained at Taconic Biosciences, USA. The TF3754 model was originally generated at Lexicon Pharmaceuticals (The Woodlands, Tex., USA) through insertion of a lacZ/Neo cassette targeting exons 2 through 3 of Gfral (NM_205844) via homologous recombination. Animals used in the described studies were a mixed background of 129S5:C57Bl/6NTac backcrossed at least once to C57Bl/6NTac mice (˜N2 B6). Adult mice were transported from Taconic Biosciences to Janssen R&D, Spring House, Pa., where they were allowed at least one week of acclimatization. All animals used in this study were maintained in accord with the protocols approved by the Institutional Animal Care & Use Committee (IACUC) at Janssen R&D, Spring House, Pa. Mice were housed on paper bedding in a temperature and humidity controlled room with 12-hour light/dark cycle and plastic enrichment. Mice were allowed ad libitum access to water and maintained on Laboratory Rodent Diet #5001 (LabDiet, USA). Food intake was measured using the BioDAQ food intake monitoring system (Research Diets, NJ, USA). Mice were singly housed on paper bedding and acclimated in the BioDAQ cages no less than 72 hours prior to the subcutaneous administration of 4 ml/kg of either PBS or recombinant human GDF15, 4 nmol/mL in PBS (generated by Janssen BioTherapeutics).

Genotyping

Tail snip DNA was used to determine the genotype of the mice. DNA extraction and amplification was performed following the REDExtract-N-Amp™ Tissue PCR Kit Protocol (Sigma-Aldrich) using the following primer sequences: TF3754—16 (SEQ ID NO: 4), TF3754—15 (SEQ ID NO:5), and Neo3b (SEQ ID NO:6). PCR products were separated by electrophoresis on a 2% agarose gel. Amplification of the wildtype allele (TF3754—16 and TF3754—15) resulted in a product of 133 base pairs while amplification of the targeted Gfral allele (Neo3b and TF3754—15) resulted in a product of 330 base pairs.

Gene Expression

To determine the expression pattern of Gfral, tissues were isolated from 5 month old male C57Bl/6N mice obtained from Taconic Biosciences, USA and included cerebellum, hindbrain, midbrain, hypothalamus, hippocampus, cortex, pituitary gland, white adipose depots, brown adipose, pancreas, liver, skeletal muscle, spleen, kidney, heart, lung, testis, stomach, regions of the small intestine, colon, thymus, adrenal gland, mesenteric lymph node, bone marrow, seminal vesicle, and epididymis. Mouse RNA was extracted with the RNeasy Lipid Tissue Mini Kit (Qiagen) with tissue homogenization performed using a TissueLyser with 5 mm stainless steel beads (Qiagen). Quantitative PCR was completed on a ViiA™ 7 Real-Time PCR System (Thermo Fisher Scientific) using TaqMan® Gene Expression Master Mix and TaqMan® Gene Expression for mouse Gfral and 18S. Relative quantity of gene expression was determined based on the back calculation to a standard curve of amplification of each gene generated from a serial dilution of pooled mouse brain cDNA containing all target genes of interest. The relative quantity of gfral was normalized to the relative quantity of 18S.

Gfral expression was analyzed using quantitative PCR on RNA obtained from brain tissues of Gfral knock-out mice as compared to littermate controls. RNA was extracted with the RNeasy Lipid Tissue Mini Kit (Qiagen) with tissue homogenization performed using a TissueLyser with 5 mm stainless steel beads (Qiagen). cDNA was prepared using the High Capacity cDNA Kit (Applied Biosystems). Quantitative PCR was completed on a ViiA™ 7 Real-Time PCR System (Thermo Fisher Scientific) using SYBR® Gene Expression Master Mix and the following primer sequences specifically targeting the junction between exons 2 and 3; mGfral Forward (SEQ ID NO:7), mGfral Reverse (SEQ ID NO:8), and mARBP Forward (SEQ ID NO:9) and mARBP Reverse (SEQ ID NO:10).

Results

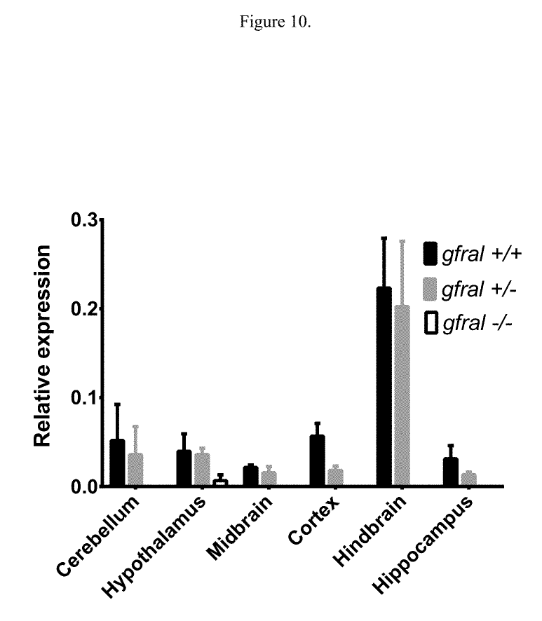

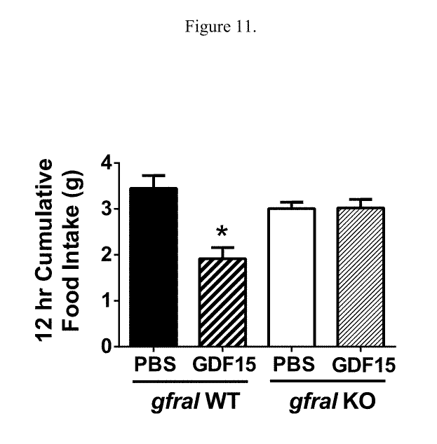

Detection of gfral expression in the mouse was limited to the brain and specifically enriched in the hindbrain (FIG. 10). The function of GFRAL in vivo was studied using mice lacking the receptor. Deletion of the receptor was confirmed by quantitative PCR (FIG. 10). To assess the dependency of GDF15 signaling on GFRAL in vivo, food intake in male GFRAL KO mice and wild type littermates was measured after treatment with recombinant human GDF15. A single subcutaneous administration of GDF15 at 16 nmol/kg lowered subsequent 12-hour food intake in the wild type mice by over 40% and this effect was absent in the GFRAL KO mice (FIG. 11).

### Example 7. Binding of HSA-GDF15 to Cynomolgus Monkey GFRAL

HSA-GDF15 was designed as HSA (SEQ ID NO:23) fused to the N-terminus of mature GDF15 (AA 197-308) via a linker (SEQ ID NO:26). HSA-GDF15 was expressed in Expi293™ cells by transient transfection and purified by CaptureSelect resin, followed by size-exclusion chromatography (SEC). Briefly, cell supernatants were loaded onto a pre-equilibrated (PBS, pH 7.2) HSA CaptureSelect column (CaptureSelect Human Albumin Affinity Matrix, ThermoFisher Scientific). After loading, unbound proteins were removed by washing the column with 10 column volumes (CV) of PBS pH 7.2. The HSA-GDF15 that was bound to the column was eluted with 10 CV of 2-M MgCl2 in 20-mM Tris, pH 7.0. Peak fractions were pooled, filtered (0.2 μm) and dialyzed against PBS, pH 7.2, at 4° C. After dialysis, the protein was filtered (0.2 μm) again and concentrated to an appropriate volume before loading onto a 26/60 superdex 200 column (GE Healthcare). Protein fractions that eluted from the SEC column with high purity (as determined by SDS-PAGE) were pooled.

To generate soluble recombinant GFRAL, the extracellular domain (ECD) of either human GFRAL (SEQ ID NO: 19) or cynomolgus monkey GFRAL (SEQ ID NO: 27) was designed to fuse to human IgG1 Fc with 6×His-tag at the C terminus (see SEQ ID NO: 28 for human and SEQ ID NO: 29 for cynomolgus monkey fusions). The GFRAL ECD proteins were expressed in Expi293™ cells by transient transfection using ExpiFectamine™ 293 transfection kit according to the manufacturer's protocol, and were purified by immobilized metal ion affinity chromatography (IMAC) followed by size-exclusion chromatography (SEC). Briefly, for each protein, the clarified cell supernatant was applied to a HisTrap HP column, followed by a stepwise elution with increasing imidazole concentration (10-500 mM). Fractions containing GFRAL ECD were identified by SDS-PAGE and pooled. The protein was filtered using a 0.2 μm membrane and concentrated to an appropriate volume before loading onto a HiLoad 26/60 Superdex 200 pg column (GE Healthcare) equilibrated with 1×DPBS, pH 7.2. Protein fractions eluted from the SEC column with high purity (determined by SDS-PAGE) were pooled and stored at 4° C.

Affinity measurements using Surface Plasmon Resonance (SPR) were performed using a ProteOn XPR36 system (Bio-Rad, Hercules, Calif.). A biosensor surface was prepared by coupling anti-Human IgG Fc (Jackson Immuno Research Labs, West Grove, Pa.) to the modified alginate polymer layer surface of a GLC chip (BioRad) using the manufacturer's instructions for amine-coupling chemistry. Approximately 5000 RU (response units) of mAbs were immobilized. The kinetic experiments were performed at 25° C. in running buffer (DPBS+0.01% P20+100 μg/ml BSA). To perform binding kinetic experiment of HSA-GDF15, GFRAL ECD-Fc or isotype control were captured followed by injections of ligands at 5 concentrations (in a 4-fold serial dilution). The association phase was monitored for 3 minutes at 50 μl/min, then followed by 15 minutes of buffer flow (dissociation phase). The chip surface was regenerated with two 18 second pulses of 100 mM H3PO4 (Sigma, St. Louis, Mo.) at 100 μL/min. The collected data were processed using ProteOn Manager software. First, the data was corrected for background using inter-spots. Then, double reference subtraction of the data was performed by using the buffer injection for analyte injections. The kinetic analysis of the data was performed using a Langmuir 1:1 binding model. The result was reported in the format of ka (On-rate), kd (Off-rate) and KD (equilibrium dissociation constant).

Binding kinetics were tested by a ProteOn SPR assay to determine the interaction between HSA-GDF15 to human and cyno GFRAL receptor extracellular domain fused to human IgG1 Fc (GFRAL ECD-Fc). Table 4 showed the binding affinity of HSA-GDF15 molecules to cyno GFRAL is within 2-fold difference compared to human GFRAL (Table 3).

TABLE 3
Summary of Binding Kinetics and Affinity for HSA-GDF15 binding to
Human GFRAL ECD-Fc

| | ka(1/Ms) 10⁶ | kd (1/s) 10⁻⁰⁴ | KD (pM) |
|---|---|---|---|
| HSA-GDF15 | 1.81 ± 0.07 | 1.56 ± 0.30 | 86.1 ± 17.8 |

TABLE 4
Summary of Binding Kinetics and Affinity for HSA-GDF15 binding to
cyno GFRAL ECD-Fc

| | ka (1/Ms) 10⁶ | kd (1/s) 10⁻⁰⁴ | KD (pM) |
|---|---|---|---|
| HSA-GDF15 | 1.86 ± 0.06 | 2.91 ± 0.30 | 157 ± 19 |

1. A method of screening compounds for having GDF15 agonistic or antagonistic activity whereby said compounds have the ability to induce or reduce GFRAL-mediated signaling.

2. (canceled)

3. The method of claim 1, wherein the method comprises the following steps:
(a) contacting a cell comprising GFRAL with a test compound;
(b) contacting a control cell, lacking the expression of GFRAL protein, with the test compound;
(c) measuring levels of GDF15 biological activity in the test cell and in the control cell;
(d) comparing the levels of GDF15 biological activity in the presence of the test compound in the test cell and in the control cell,
wherein an increase in the levels of the GDF15 biological activity in the test cell, relative to that in the control cell, indicates that the test compound has GDF15 agonistic activity,
and wherein a decrease in the levels of the GDF15 biological activity in the test cell, relative to that in the control cell, indicates that the test compound has GDF15 antagonistic activity.

4. The method of claim 1, wherein the method comprises the following steps:
(a) contacting a test animal, expressing GFRAL protein, with a test compound;
(b) contacting a control animal, lacking the expression of GFRAL protein with the test compound;
(c) measuring body weight or food intake in the test animal and the control animal;
(d) comparing the body weight or food intake in the presence of the test compound in the test animal and the control animal,
wherein the decrease in the body weight or food intake in the test animal relative to that in the control animal, indicates that the test compound has GDF15 agonistic activity;
and wherein the increase in the body weight or food intake in the test animal relative to that in the control animal, indicates that the test compound has GDF15 antagonistic activity.

5. The method of claim 3 wherein the GDF15 biological activity comprise phosphorylation of tyrosine.

6. The method of claim 3 wherein the GDF15 biological activity comprise phosphorylation of Akt.

7. The method of claim 3 wherein the GDF15 biological activity comprise phosphorylation of Erk1/2.

8. The method of claim 3 wherein the GDF15 biological activity comprise phosphorylation of PLCγ1.

9. The method of claim 3 wherein measuring the levels of the GDF15 biological activity comprise measuring levels of a reporter signal.

10. The method of claim 3 wherein the compound is a part of a library of compounds.

11. The method of claim 3 wherein the compound is a composition.

12. The method of claim 3 wherein the compound is a fusion protein.

13. The method of claim 3 wherein GFRAL comprises a sequence having at least 94% identity to human GFRAL extracellular domain sequence.

14. A kit for screening test compounds for having GDF15 agonistic activity, comprising a cell capable of expressing GFRAL protein and instructions for using the kit in a method for screening test compounds for having GDF15 agonistic activity.

15. The kit of claim 14, wherein the cell capable of expressing GFRAL protein is a stably or transiently transfected cell.

16. A kit for screening test compounds for having GDF15 antagonistic activity, comprising a cell capable of expressing GFRAL protein and instructions for using the kit in a method for screening test compounds for having GDF15 antagonistic activity.

17. The kit of claim 16, wherein the cell capable of expressing GFRAL protein is a stably or transiently transfected cell.

18. A method of treating a metabolic disorder, comprising administering to a subject a therapeutically effective amount of a compound identified by the method of claim 1.

19. The method of claim 18 wherein the metabolic disorder is selected from the group consisting of type 2 diabetes, hyperglycemia, hyperinsulinemia, obesity, dyslipidemia, and diabetic nephropathy.


SEQUENCE LISTING (25 sequences)

SEQ ID NO: 1 - protein, 339 aa, artificial
  GDF15-Fc fusion protein
CPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGKGSGGGARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 2 - protein, 222 aa, artificial
  Fc
CPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 394 aa, human
MIVFIFLAMGLSLENEYTSQTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGEVIYAAMCMTVTCGILLLVMVKLRTSRISSKARDPSSIQIPGEL

SEQ ID NO: 4 - DNA, 24 bp, artificial
  PCR primer
gtatggatgacctccactgtacag

SEQ ID NO: 5 - DNA, 30 bp, artificial
  PCR primer
caacaaatgaacacatattgattgagtagg

SEQ ID NO: 6 - DNA, 23 bp, artificial
  PCR primer
gtgtggcggaccgctatcaggac

SEQ ID NO: 7 - DNA, 20 bp, mouse
gctgtgagcagtcatggaga

SEQ ID NO: 8 - DNA, 20 bp, mouse
ttccaccaaagcctggatac

SEQ ID NO: 9 - DNA, 18 bp, mouse
ggcaccgaggcaacagtt

SEQ ID NO: 10 - DNA, 21 bp, mouse
tcatccagcaggtgtttgaca

SEQ ID NO: 11 - protein, 19 aa, artificial
  signal peptide
MAWVWTLLFLMAAAQSIQA

SEQ ID NO: 12 - protein, 8 aa, artificial
  flag tag
DYKDDDDK

SEQ ID NO: 13 - protein, 376 aa, human
SQTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGEVIYAAMCMTVTCGILLLVMVKLRTSRISSKARDPSSIQIPGEL

SEQ ID NO: 14 - protein, 441 aa, human
DRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISDVFQQVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS

SEQ ID NO: 15 - protein, 443 aa, human
SPSSLQGPELHGWRPPVDCVRANELCAAESNCSSRYRTLRQCLAGRDRNTMLANKECQAALEVLQESPLYDCRCKRGMKKELQCLQIYWSIHLGLTEGEEFYEASPYEPVTSRLSDIFRLASIFSGTGADPVVSAKSNHCLDAAKACNLNDNCKKLRSSYISICNREISPTERCNRRKCHKALRQFFDRVPSEYTYRMLFCSCQDQACAERRRQTILPSCSYEDKEKPNCLDLRGVCRTDHLCRSRLADFHANCRASYQTVTSCPADNYQACLGSYAGMIGFDMTPNYVDSSPTGIVVSPWCSCRGSGNMEEECEKFLRDFTENPCLRNAIQAFGNGTDVNVSPKGPSFQATQAPRVEKTPSLPDDLSDSTSLGTSVITTCTSVQEQGLKANNSKELSMCFTELTTNIIPGSNKVIKPNSGPSRARPSAALTVLSVLMLKLAL

SEQ ID NO: 16 - protein, 370 aa, human
GDPLPTESRLMNSCLQARRKCQADPTCSAAYHHLDSCTSSISTPLPSEEPSVPADCLEAAQQLRNSSLIGCMCHRRMKNQVACLDIYWTVHRARSLGNYELDVSPYEDTVTSKPWKMNLSKLNMLKPDSDLCLKFAMLCTLNDKCDRLRKAYGEACSGPHCQRHVCLRQLLTFFEKAAEPHAQGLLLCPCAPNDRGCGERRRNTIAPNCALPPVAPNCLELRRLCFSDPLCRSRLVDFQTHCHPMDILGTCATEQSRCLRAYLGLIGTAMTPNFVSNVNTSVALSCTCRGSGNLQEECEMLEGFFSHNPCLTEAIAAKMRFHSQLFSQDWPHPTFAVMAHQNENPAVRPQPWVPSLFSCTLPLILLLSLW

SEQ ID NO: 17 - protein, 279 aa, human
VGGNRCVDAAEACTADARCQRLRSEYVAQCLGRAAQGGCPRARCRRALRRFFARGPPALTHALLFCPCAGPACAERRRQTFVPSCAFSGPGPAPPSCLEPLNFCERSRVCRCARAAAGPWRGWGRGLSPAHRPPAAQASPPGLSGLVHPSAQRPRRLPAGPGRPLPARLRGPRGVPAGTAVTPNYVDNVSARVAPWCDCGASGNRREDCEAFRGLFTRNRCLDGAIQAFASGWPPVLLDQLNPQGDPEHSLLQVSSTGRALERRSLLSILPVLALPALL

SEQ ID NO: 18 - DNA, 9 bp, artificial
  Kozak sequence
gccgccacc

SEQ ID NO: 19 - protein, 333 aa, human
SQTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGE

SEQ ID NO: 20 - protein, 15 aa, artificial
  Avi tag
GLNDIFEAQKIEWHE

SEQ ID NO: 21 - protein, 308 aa, human
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 22 - protein, 10 aa, artificial
  FLAG tag
EFDYKDDDDK

SEQ ID NO: 23 - protein, 584 aa, human
AHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 24 - protein, 22 aa, artificial
  genetic linker
GSGGGGSGGGGSGGGGSGGGGS

SEQ ID NO: 25 - protein, 5 aa, artificial
  genetic linker
GSGGG